(19) United States
(12) Patent Application Publication
(10) Pub. No.: US 2007/0082863 A1
(43) Pub. Date: Apr. 12, 2007

(54) Methods and compositions for maturing dendritic cells utilizing inosine-containing compounds

(76) Inventors: Kathy L. Signorelli, Kings Park, NY (US); James E. Egan, Stony Brook, NY (US); Frances Santiago-Schwarz, Cold Springs Harbor, NY (US)

Correspondence Address: KOHN & ASSOCIATES PLLC, 30500 NORTHWESTERN HWY, STE 410, FARMINGTON HILLS, MI, 48334, US

(21) Appl. No.: 10/554,688

(22) PCT Filed: Apr. 27, 2004
(86) PCT No.: PCT/US04/13141

§ 371 Date: Oct. 16, 2006

Related U.S. Application Data
(60) Provisional application No. 60/466,700, filed on Apr. 29, 2003.

(51) Int. Cl. A61K 48/00; A61K 31/7076

(52) U.S. Cl. 514/44; 514/45

(57) ABSTRACT

A method of stimulating in vivo or ex vivo maturation of dendritic cells by applying an effective amount of inosine-containing compounds to the dendritic cells. The maturation of dendritic cells leads to more robust cellular immune responses against antigens including those associated with vaccines, infectious agents, and tumor cells by enhancing the stimulatory activity of dendritic cells toward T cells. An in vivo or ex vivo method of treating diseases in a subject by applying an effective amount of an inosine-containing compound to the dendritic cells to stimulate maturation thereof; and administering matured dendritic cells into the subject. A method of enhancing the immune response of a host mammal by isolating immature dendritic cells from a donor mammal; maturing the immature dendritic cells in the presence of an inosine-containing compound ex vivo; and administering the mature dendritic cells to a host mammal in an amount effective to enhance the immune response of the host mammal. A method of maturing dendritic cells in vivo or ex vivo in the presence of an inosine-containing compound, which results in increasing the stimulation of T cells in response to an antigen. A method of enhancing the immunological response to vaccine antigens by inclusion of an inosine-containing compound. A composition for in vivo or ex vivo maturation of dendritic cells including an inosine-containing compound, wherein one possible form of the inosine-containing compound is an IpR oligonucleotide molecule. Compositions, pharmaceutical composition, adjuvants, immunostimulants, and kits for maturing dendritic cells in vivo or ex vivo including an inosine-containing compound.

### BACKGROUND OF THE INVENTION

1. Technical Field

The present invention relates to methods of enhancing immune response in a host mammal. Specifically, the present invention relates to methods of increasing the maturity, functionality, and effectiveness of dendritic cells.

2. Background Art

Protective immunity results from the joint actions of both innate and adaptive immunity. Adaptive immunity, which is mediated by B and T lymphocytes, is characterized by highly specific recognition of pathogen derived components via antigen-specific receptors and the generation of immunological “memory.” The establishment of adaptive immunity takes time to develop and is not in place for days to weeks following exposure to a microbe. In contrast, innate immunity responds rapidly through the recognition of conserved, rather than unique, structural determinants of the pathogen with a set of defined antimicrobial strategies including the production of inflammatory cytokines and phagocytosis. Innate immunity involves various cell components such as natural killer cells, monocyte, macrophages, granulocytes, neutrophils, and dendritic cells. Innate immunity is important in host defense during early stages of infection. In addition, innate immunity, primarily via dendritic cell signaling, drives and directs subsequent adaptive responses by producing specific “instructional” cytokines and interactions of co-stimulatory molecules with T cells. Thus, dendritic cells play a pivotal role in both early and late responses against introduced pathogens.

Dendritic cells, found in virtually every tissue and organ of the body, are antigen presenting cells that regulate a wide spectrum of responses within both the adaptive immune response (including Th1, Th2, CD8 and B cell responses) as well as influencing the innate immune system. Dendritic cells include a complex system of cells encompassing multiple subsets and distinct biological functions, which vary with both their lineage and stage of differentiation.

There are three distinct human subpopulations of dendritic cells, originating from two distinct lineages of hematopoietic progenitors: (1) myeloid and (2) lymphoid. Dendritic cell subsets and maturation stages are defined by a combination of markers (See, FIG. 1). Progression down a given pathway is driven by particular cytokines and recent advances in culturing techniques have allowed many of the various subtypes and maturation levels to be grown in vitro (typically from either CD34+ bone-marrow or cord blood cells or from peripheral blood).

Within the myeloid lineage, two developmental pathways are possible. One yields the monocyte-derived dendritic cell (also known as the CD14-derived, DC1, or M-DC). Precursors for M-DC are present in peripheral blood and can be cultured in vitro, typically with GM-CSF and IL-4. Once committed to differentiate toward a dendritic cell rather than a monocyte, they are recognized as CD14dim CD1b/c+. Upon complete maturation, these cells secrete significant amounts of IL-12 and as such, are responsible for polarizing T cells toward Th1 type responses, hence the designation as DC1.

The second myeloid dendritic cell pathway produces a CD14-independent Langerhans cell dendritic cell subtype whose differentiation is critically dependent on the presence of TGFβ.

The third subpopulation is the lymphoid-derived plasmacytoid dendritic cell (also known as DC2 or P-DC). The P-DC is derived from an immediate precursor that exhibits a plasma cell-like morphology. P-DC precursors can also be found in peripheral blood using newly described markers BDCA-2 and 4 and their growth in vitro is dependent on IL-3+/− CD40 ligand (CD40L). This dendritic cell subtype was originally thought to promote the development of Th2 T cells due to a lack of IL-12 production; however, more recently it has been identified as the primary producer of type I interferons (IFNα and β) and produce small amounts of IL-12 with appropriate stimulation.

While the primary function of both P-DC and M-DC is to act as antigen presenting cells, they do have somewhat different functional capabilities in terms of the types of cytokines and chemokines they secrete in response to stimuli, as well as their abilities to foster various types of differentiation in T cells, i.e. Th1, Th2, Treg, etc. These differences in functional capacity are in part related to alternative expression of various receptors that initially recognize the foreign pathogen or “danger” signal. Such receptors include the Toll-like receptors (TLRs), heat shock protein receptor (CD91), scavenger receptors, mannose and other lectin receptors and receptors for complement. For example, P-DC only express TLR 7 and 9, which bind to imidazoquinolines and CpG motifs respectively. M-DC express TLR1-6, which bind to assorted bacterial cell wall components (e.g. LPS, peptidoglycan, flagellin, etc.) and viral elements (e.g. ds RNA).

The functions of a dendritic cell are different depending upon its lineage and state of maturation. Immature dendritic cells are present in peripheral tissues or circulating In blood where they continuously sample the antigenic environment. Generally speaking, immature dendritic cells, are good at picking up foreign materials/pathogens and digesting them, however, they are not particularly good at presenting antigens to T cells in a stimulatory fashion. Upon an encounter with microorganisms, microbial products, or tissue damage (collectively referred to as “danger signals”), dendritic cells initiate their differentiation to a mature phenotype, including processing and presenting a sampling of antigens on their surface through increased surface expression of Class I and Class II peptide-major histocompatibility complexes. The dendritic cells concomitantly migrate to the lymph nodes, mediated by a change in chemokine receptor expression. Additionally, the dendritic cells upregulate expression of co-stimulatory molecules (CD86, CD80, etc.), which are required for effective interactions with T cells.

Thus, upon maturation, dendritic cells become less adept at antigen uptake and better at presentation to T cells including expression of increased MHC Class I and Class II molecules, as well as a variety of co-stimulatory molecules, e.g. CD80, CD86. In addition, dendritic cells interact with a wide variety of cellular and non-cellular components of the innate immune system. Influences on natural killer cells and other innate cell types are mediated by mature dendritic cells typically by the production of activating cytokines (e.g., IL-12, IFNα/β, TNF, and IL-1) and chemokines, (e.g., interleukin 8 (IL8)). However, direct interactions via surface molecules such as CD1 can also occur.

In standard practice, human peripheral blood monocyte-derived dendritic cell precursors are isolated by a process, which involves adherence of cells from a blood mononuclear cell preparation to tissue culture dishes for about ninety minutes, followed by culture with the cytokines GM-CSF and IL4 for a period of 6-7 days. At this point, a second “danger” or pathogen derived signal, such as TNF or LPS, is added to stimulate the final maturation steps, which can take up to another six days of culture. It is important to note that differentiation to a fully mature state in vitro and in vivo requires the second “danger signal” such as that from a viral or bacterial product such as LPS. This final maturation step is correlated with increased antigen presentation, expression of costimulatory molecules, cytokine and chemokine secretion, and subsequent stimulation of na [symbol] ve T cells, all of which are crucial to effective pathogen protection.

Appropriate recognition of microbial danger and cellular stress is vital to survival of the host as this leads to activation of local defense mechanisms and recruitment and activation of specialized immune cells. Thus, dendritic cells as well as other cells of the innate immune system have evolved a variety of means for doing so, using so called “pattern recognition receptors” (PRRs). The PRRs recognize molecular patterns (pathogen-associated molecular patterns or PAMPs) in non-processed antigens such as cell wall components or nucleic acids of pathogens that are shared by large groups of microorganisms, but are distinct from those found in the host. Dendritic cells express PRRs including CD14, mannose receptor, DEC 205, and the family of toll-like receptors (TLRs).

In the prior art, substances typically of microbial origin had been used to non-specifically stimulate immune responses (e.g., inclusion of mycobacteria in Freund's adjuvant). With advances in understanding of innate immune responses and dendritic cells in particular, it has become clear that most, if not all of these substances act on dendritic cells. Several recent publications disclose the use of immunostimulatory oligonucleotides containing an unmethylated CpG motif (CpGs) and two synthetic imidazoquinoline compounds (Resiquimod and Imiquimod). As described above, the specific TLR used by a given compound have been identified (i.e., TLR9 recognizes CpGs). Because of the mutually exclusive expression of specific TLRs to either the P-DC or M-DC lineages (e.g., restriction of TLR9 to the P-DC lineage), this implies that the functional consequences of these compounds may be limited to the functional repertoire of a given lineage. An ideal broad-spectrum anti-pathogen agent might show wider targeting to several innate cell types.

Accordingly, there is a need for a compound and related method that induces maturation of dendritic cells for a more productive immunogenic response.

### SUMMARY OF THE INVENTION

The present invention provides for compositions, kits, and methods of enhancing the maturation of dendritic cells in vivo or ex vivo. The present invention provides for the in vivo or ex vivo stimulation of dendritic cells to a mature phenotype. More specifically, the present invention provides for a method of stimulating in vivo or ex vivo maturation of dendritic cells by applying an effective amount of inosine-containing compounds to the dendritic cells. Additionally, the present invention provides for a method of treating diseases in a subject by applying an effective amount of an inosine-containing compound to the dendritic cells to stimulate maturation thereof; and administering matured dendritic cells into the subject. Further, there is provided a method of enhancing the immune response of a host mammal by isolating immature dendritic cells from a donor mammal; maturing the immature dendritic cells in the presence of an inosine-containing compound either in the presence or absence of antigen(s) and administering the mature dendritic cells to a host mammal in an amount effective to enhance the immune response of the host mammal. The present invention also provides for a method of maturing dendritic cells in vivo or ex vivo in the presence of an inosine-containing compound, which results in increasing presentation of the antigens to T-cells in a stimulatory fashion. The present invention provides combining antigens with an inosine-containing compound to be administered in the form of a vaccine thereby enhancing the response to the vaccine antigens wherein the inosine-containing compound is considered as an adjuvant. Finally, the present invention provides for a composition for in vivo or ex vivo maturation of dendritic cells including an inosine-containing compound.

### BRIEF DESCRIPTION OF THE DRAWINGS

Other advantages of the present invention will be readily appreciated as the same becomes better understood by reference to the following detailed description when considered in connection with the accompanying drawings wherein:

FIG. 1 illustrates dendritic cell populations from human peripheral blood;

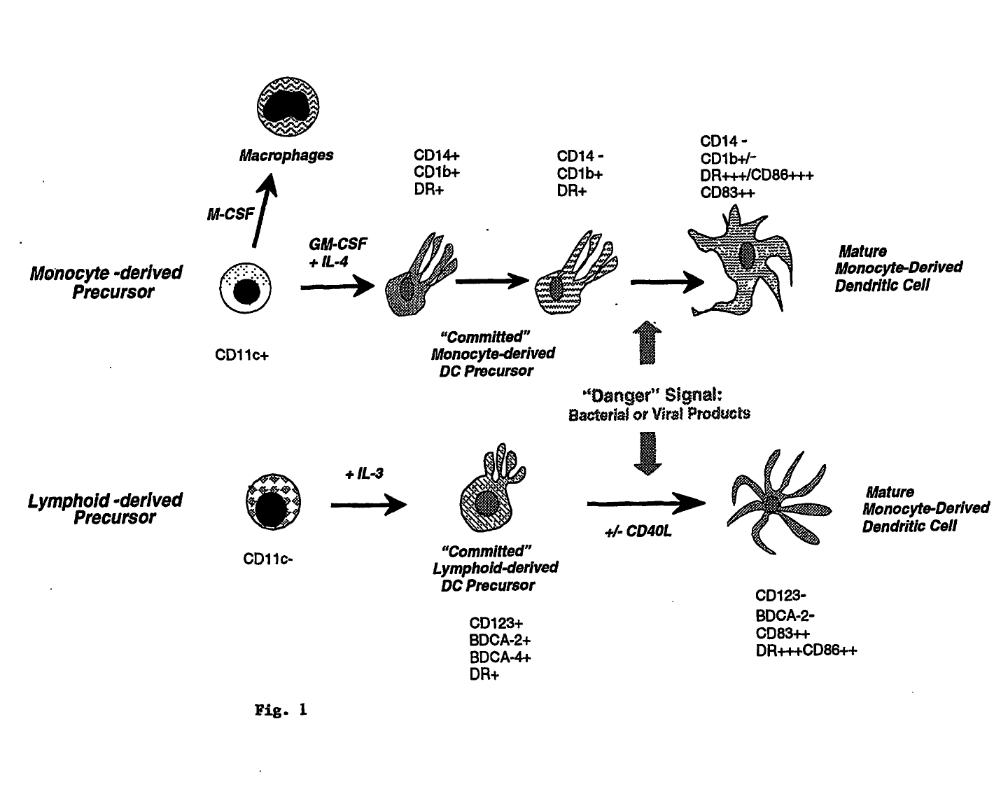

FIG. 2 demonstrates the effect of pre-treatment of mice (lethally challenged with Listeria monocytogenes) with orally administered MIMP to extend survival time;

FIG. 3 demonstrates that MIMP extends survival in a Friend Leukemia Virus (FLV) lethal challenge mouse model, wherein 6-8 week old female mice were infected i.p. with 0.2 ml FLV stock causing 100% mortality by 30-45 days (mean survival time for controls was 39 days) compared to a mean survival for MIMP-treated mice of 46 days, which is an approximate 18% increase);

FIG. 4 is a bar graph illustrating that MIMP acts as an adjuvant when combined with an immunizing antigen (inactivated influenza) to increase a T-cell mediated delayed type hypersensitivity (DTH) response to that antigen;

FIG. 5A shows two photographs that are 40× magnification Wright stained cytospins illustrating that MIMP induces morphological maturation of M-DCs and FIG. 5B (**MFI=Mean Fluorescence Intensity) is a chart showing MIMP induced changes in cell surface markers associated with dendritic cell maturation;

FIG. 6 shows three histograms generated by flow cytometry illustrating that MIMP induces a recognized dendritic cell maturation marker, CD83 on human peripheral blood adherent mononuclear cells, cultured with the indicated amount of MIMP;

FIG. 7 is a bar graph illustrating that MIMP increases the functional maturation of dendritic cells as demonstrated by enhanced stimulation of na [symbol] ve T cells; and

FIG. 8 is a bar graph illustrating that MIMP treatment of human dendritic cells derived from adherent peripheral blood mononuclear cells, augments production of the chemoattractant IL8 above the amount made in the presence of GM-CSF and IL4 alone, and which is significantly enhanced by day 7 of in vitro culture.

### DETAILED DESCRIPTION OF THE INVENTION

Generally, the present invention is directed towards compositions, methods, and kits for accelerating the maturation of dendritic cells in vivo or ex vivo through the application of inosine-containing compounds. The present invention is also useful in activating an individual's T cells by administering the primed dendritic cells to the individual, or activation of T cells in vitro by virtue of co-incubation with the dendritic cells matured with an inosine-containing compound, which are then administered to the individual. The present invention is also useful in priming dendritic cells in vivo. The present invention is also useful for enhancing the immunological responses to vaccines by acting as a dendritic cell stimulating adjuvant.

The term “dendritic cells” as used herein is defined as antigen-presenting cells in the body that are responsible for priming na [symbol] ve T cells to respond to a specific antigen, whereby the T cell further differentiates into an “effector” cell, which can have functions such a T helper cell or cytotoxic T cell or into a “memory” T cell. Dendritic cells also secrete a variety of cytokines and chemokines, which stimulate and direct T cell function as well as stimulating other immune cells including innate immune system cells such as natural killer cells, which provide immediate, non-pathogen specific killing of pathogens. Dendritic cells include, but are not limited to, plasmacytoid dendritic cells (hereinafter, “P-DC”) and myeloid or monocyte dendritic cells (hereinafter, “M-DC”).

The term “inosine-containing compounds” as used herein means any compound that includes an inosine molecule. Such inosine molecules include, but are not limited to, Isoprinosine, inosine 5′-monophosphate, Methyl inosine 5′-monophosphate (hereinafter, “MIMP”), polymers thereof such as dimers and trimers, homologues thereof, derivatives thereof, and any inosine-containing compound known to those of skill in the art. Inosine-containing compounds enhance the immune response of the individual to the antigen or compound by making the immune system more responsive. The inosine-containing compound also affects the immune response such that a lower dose of the antigen or compound is required to achieve an immune response in the individual. The inosine-containing compound can also be an oligonucleotide bonded to or containing an inosine molecule through a phosphate bond or —S group.

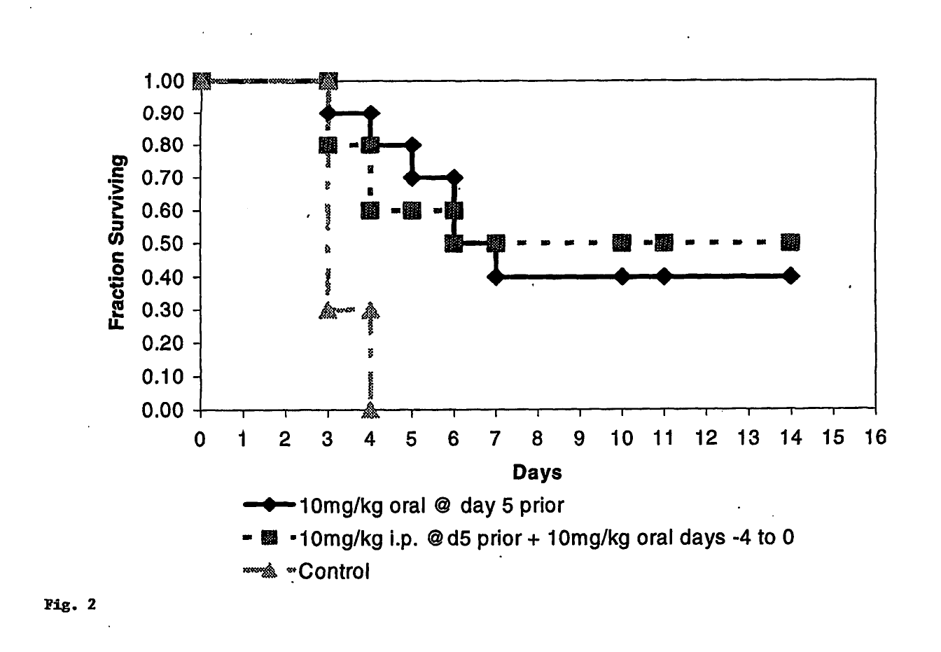

In the preferred embodiment, MIMP is utilized as the inosine-molecule. MIMP is a synthetic analog of the naturally occurring purine nucleoside inosine monophosphate (more specifically, inosine 5′-monophosphate). In vitro and in vivo studies to date have shown that the immunostimulating activity of MIMP primarily targets T cell-dependent immune responses and preferentially enhances cell-mediated immune function (See, FIGS. 2-4).

Inosine 5′-monophosphate is an important purine that has great immunopotentiating capabilities. Inosine 5′-monophosphate, specifically MIMP, is described in U.S. Pat. No. 5,614,504 to Hadden et al., which is incorporated herein by reference. This immunomodulator is effective in the treatment of infections of intracellular bacterial pathogens and viruses. Inosine 5′-monophosphate has the general formula:

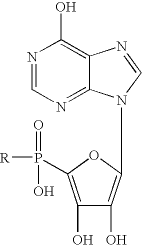

wherein said R-group is a moiety selected from the group consisting of alkyl, alkoxy, arginine, secondary amino compounds, —OCH₃ (to form MIMP), and the like. The R-group has numerous functions. For example, the R-group has protective function such as inhibiting hydrolysis of MIMP by enzymes such as 5′-nucleotidase, phosphodiesterases, and the like.

The inosine-5′-monophosphate derivatives are enzyme resistant (“protected-IMP”) and are immunopotentiators. These protected derivatives of inosine-5′-monophosphate as described herein can be readily prepared by condensation of a desired alcohol, primary amine, or peptide with inosine-5′-monophosphate, preferably in the presence of a condensing agent such as dicyclohexylcarbodiimide or the like. Suitable alcohols include monohydric alcohols of 1 to 20 carbon atoms such as methyl alcohol, ethyl alcohol, n-propyl alcohol, n-butyl alcohol, n-hexyl alcohol, n-octyl alcohol, and n-decyl alcohol.

The terms “cell surface markers,” “costimulatory molecules,” “cell surface receptors,” “receptors,” and “cell receptors” as used herein is defined as cell membrane glycoproteins that are partially or fully exposed on the outside surface of the cell and interact with other structures, molecules, or proteins. Cell surface markers include, but are not limited to, CD1a-c, CD11c, CD14, CD40, CD80, CD83, CD86, CD123, HLA-DR, BDCA-2, BDCA-4, Toll-like receptors (TLR), heat shock protein receptors (CD91), scavenger receptors, mannose receptors, complement receptors, lectin receptors, and any other cell surface markers or receptors known to those of skill in the art.

The term “effective amount” as used herein means an amount that is determined by such considerations as are known in the art of treating secondary immunodeficiencies wherein it must be effective to provide measurable relief in treated individuals, such as exhibiting improvements including, but not limited to, improved survival rate, more rapid recovery, improvement or elimination of symptoms, reduction of post infectious complications and, where appropriate, antibody titer or increased titer against the infectious agent, reduction in tumor mass, and other measurements as known to those skilled in the art.

The term “antigen” as used herein is defined as any material that can be specifically bound by an antibody, T-cell receptors, or pattern recognition receptors (PRRs), thereby inducing an immune response. Types of antigens include, but are not limited to viral, bacterial, tumor and self-antigens. Accordingly, the dendritic cells prepared according to this invention are useful for the prevention and treatment of various diseases including infectious disease, cancer, autoimmune disease, and bioterrorism.

The terms “nucleic acid” and “oligonucleotide” are used interchangeably and are defined as multiple nucleotides (i.e., molecules comprising a sugar (e.g., ribose or deoxyribose) linked to a phosphate group and to an exchangeable organic base, which is either a substituted pyrimidine (e.g., cytosine (C), thymine (T) or uracil (U)) or a substituted purine (e.g., adenine (A), guanine (G), or inosine (I)). The terms refer to both oligoribonucleotides and oligodeoxyribonucleotides. The terms shall also include polynucleosides (i.e., a polynucleotide minus the phosphate) and any other organic base-containing polymer. Nucleic acid molecules can be obtained from existing nucleic acid sources (e.g., genomic or cDNA), but can also be synthetic (e.g., produced by oligonucleotide synthesis).

The present invention has numerous advantages over the prior art. For example, the present invention enhances and increases the maturation of dendritic cells. As a result, elaboration of the mature functional properties of the dendritic cell is accelerated. The maturation of dendritic cells leads to more robust cellular immune responses against antigens including those associated with vaccines, infectious agents, and tumor cells by enhancing the stimulatory activity of dendritic cells toward T cells. Mature dendritic cells provide for better in vivo immune responses to vaccines and pathogens.

The present invention has numerous embodiments directed towards various methods, compositions, adjuvants, immunostimulants, and kits. In one embodiment, the present invention is directed towards a method of stimulating maturation of dendritic cells in vivo or ex vivo by applying an effective amount of inosine-containing compounds to the dendritic cells. The inosine-containing compound includes, but is not limited to, isoprinosine, inosine 5′-monophosphate, methyl inosine 5′-monophosphate (MIMP), inosine-containing oligonucleotides, polymers thereof such as dimers and trimers, oligonucleotides including one or more inosine 3′,5′ linkages, homologues thereof, and derivatives thereof.

As set forth herein, maturation of dendritic cells results in the dendritic cells being capable of stimulating na [symbol] ve T cells more effectively, to express a particular constellation of phenotypic cell surface markers, and to produce and respond to specific cytokines and chemokines. More specifically, maturation is defined as the acquisition of several properties: typical stellate morphology; upregulation of MHC molecules (Class I and Class II) and co-stimulatory molecules (CD80, CD86), and the mature DC-specific marker, CD83. Maturation is accompanied by a coordinated series of changes that include downregulation of monocytic cell markers, i.e. CD14, and decreased antigen uptake via macropinocytosis, phagocytosis or endocytosis. The combined increase in MHC and costimulatory molecules and decrease in antigen uptake are related to the mature DC's enhanced ability to stimulate na [symbol] ve T cells. Mature dendritic cells become less efficient at processing soluble antigens, but highly efficient at presenting antigens to T cells in a stimulatory fashion. DCs further modulate the activity of T cells and other Immune cell types by production of cytokines and chemokines.

Maturation of dendritic cells can be assessed by an evaluation of relevant surface markers. Such surface markers include, but are not limited to, CD1a-c, CD11c, CD14, CD40, CD80, CD83, CD86, CD123, HLA-DR, Toll-like receptors (TLRs), heat shock protein receptors (CD91), scavenger receptors, mannose receptors, complement receptors, lectin receptors, and any other cell surface markers or receptors known to those of skill in the art. The present invention also provides. for a method of treating diseases in a subject by loading dendritic cells with antigen compounds; applying an effective amount of an inosine-containing compound to the dendritic cells to stimulate maturation thereof; and administering matured dendritic cells into the subject. This method also includes the further step of fostering the secretion of cytokines and chemokines, which foster the development of Th1 responses in T cells. Administering the mature dendritic cells can occur by any means known to those of skill in the art including, but not limited to, intravenous, subcutaneous, intraperitoneal, intratumoral and peritumoral. Furthermore, the mature dendritic cells can be administered with a pharmaceutically acceptable carrier as is well known to those of skill in the art.

Another method of the present invention is a method of stimulating maturation of dendritic cells in vitro by applying an inosine-containing compound to the dendritic cells thereby increasing dendritic processes, and increasing functionality of the dendritic cells thereof.

The present invention is useful in enhancing the immune response of a host mammal. This is accomplished by isolating Immature dendritic cells from a donor mammal; maturing the immature dendritic cells in the presence of an inosine-containing compound in vitro; and administering the mature dendritic cells to a host mammal in an amount effective to enhance the immune response of the host mammal. Optionally, enhancement of the immune response can further include the step of loading the immature dendritic cells with antigens. A further method of the present invention is a method of increasing presentation of antigens to T-cells in a stimulatory fashion by maturing dendritic cells in the presence of an inosine-containing compound. This results in increased proliferation of T cells in response to the antigen (See, Examples Section).

In any of the above-described methods, the dendritic cells are incubated under various conditions. For example, in one embodiment, the dendritic cells are treated with the inosine-containing compound for about 24 hours (48 hours of total culture in the presence of GM-CSF+IL-4).

The present invention also provides for various compositions. In one embodiment, there is provided a composition for maturing dendritic cells ex vivo for treatment of various in vivo diseases. This composition is an inosine-containing compound that includes, but is not limited to, isoprinosine, inosine 5′-monophosphate, methyl inosine 5′-monophosphate (MIMP), inosine-containing oligonucleotides, polymers thereof such as dimers and trimers, oligonucleotides including one or more inosine 3′,5′ linkages, homologues thereof, and derivatives thereof. Preferably, the inosine-containing compound is in a dose rage of approximately 1 to 300 μg/ml (approximately 3 to 900 ,μM or 1 to 300 mg/kg). Further, the composition is useful in treating various diseases including, but not limited to, cancer, immune deficiencies, and any other immune related diseases known to those of skill in the art. Moreover, the composition is useful for generating enhanced T-cell immune activity for the treatment of various diseases corresponding to various infections caused by agents including, but not limited to, viruses, bacteria, influenza, HIV, hepatitis B, hepatitis C, anthrax, other pathogens, and any other infectious agents known to those of skill in the art.

There is also provided a pharmaceutical composition for improving in vivo dendritic cell function including an effective amount of an inosine-containing compound. Further, there is provided an immunostimulant for use in a vaccine comprising an inosine-containing compound for use in maturing dendritic cells, wherein antigens are of low immunogenicity and multiple doses are required. Additionally, there is provided an oral or other adjuvant to be used for vaccines including an inosine-containing compound for use in maturing dendritic cells.

The composition of the present invention can also be combined with various pharmaceutical compositions and/or components, including adjuvants. The composition of the present invention is administered and dosed in accordance with good medical practice, taking into account the clinical condition of the individual patient, the site and method of administration, scheduling of administration, patient age, sex, body weight and other factors known to medical practitioners. The pharmaceutically “effective amount” for purposes herein is thus determined by such considerations as are known in the art. The amount must be effective to achieve improvement including but not limited to improved survival rate or more rapid recovery, or improvement or elimination of symptoms and other indicators as are selected as appropriate measures by those skilled in the art.

In the method of the present invention, the compound of the present invention can be administered in various ways. It should be noted that it can be administered as the compound or as pharmaceutically acceptable salt and can be administered alone or as an active ingredient in combination with pharmaceutically acceptable carriers, diluents, adjuvants and vehicles. The compounds can be administered orally, subcutaneously or parenterally including intravenous, intraarterial, intramuscular, intraperitoneally, and intranasal administration as well as intrathecal and infusion techniques. Implants of the compounds are also useful. The patient being treated is a warm-blooded animal and, in particular, mammals including man. The pharmaceutically acceptable carriers, diluents, adjuvants and vehicles as well as implant carriers generally refer to inert, non-toxic solid or liquid fillers, diluents or encapsulating material not reacting with the active ingredients of the invention. The doses may be single doses or multiple doses over a period of several days. The treatment generally has a length proportional to the length of the disease process and drug effectiveness and the patient species being treated.

When administering the compound of the present invention parenterally, it will generally be formulated in a unit dosage injectable form (solution, suspension, emulsion). The pharmaceutical formulations suitable for injection include sterile aqueous solutions or dispersions and sterile powders for reconstitution into sterile injectable solutions or dispersions. The carrier can be a solvent or dispersing medium containing, for example, water, ethanol, polyol (for example, glycerol, propylene glycol, liquid polyethylene glycol, and the like), suitable mixtures thereof, and vegetable oils.

Proper fluidity can be maintained, for example, by the use of a coating such as lecithin, by the maintenance of the required particle size in the case of dispersion and by the use of surfactants. Nonaqueous vehicles such a cottonseed oil, sesame oil, olive oil, soybean oil, corn oil, sunflower oil, or peanut oil and esters, such as isopropyl myristate, may also be used as solvent systems for compound compositions. Additionally, various additives which enhance the stability, sterility, and isotonicity of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. In many cases, it will be desirable to include isotonic agents, for example, sugars, sodium chloride, and the like. Prolonged absorption of the injectable pharmaceutical form can be brought about by the use of agents delaying absorption, for example, aluminum monostearate and gelatin. According to the present invention, however, any vehicle, diluent, or additive used would have to be compatible with the compounds.

Sterile injectable solutions can be prepared by incorporating the compounds utilized in practicing the present invention in the required amount of the appropriate solvent with various of the other ingredients, as desired.

A pharmacological formulation of the present invention can be administered to the patient in an injectable formulation containing any compatible carrier, such as various vehicle, adjuvants, additives, and diluents; or the compounds utilized in the present invention can be administered parenterally to the patient in the form of slow-release subcutaneous implants or targeted delivery systems such as monoclonal antibodies, vectored delivery, iontophoretic, polymer matrices, liposomes, and microspheres. Examples of delivery systems useful in the present invention include: U.S. Pat. Nos. 5,225,182; 5,169,383; 5,167,616; 4,959,217; 4,925,678; 4,487,603; 4,486,194; 4,447,233; 4,447,224; 4,439,196; and 4,475,196. Many other such implants, delivery systems, and modules are well known to those skilled in the art.

A pharmacological formulation of the compound utilized in the present invention can be administered orally to the patient. Conventional methods such as administering the compounds in tablets, suspensions, solutions, emulsions, capsules, powders, syrups and the like are usable. Known techniques that deliver it orally or intravenously and retain the biological activity are preferred.

In one embodiment, the compound of the present invention can be administered initially by intravenous injection to bring blood levels to a suitable level. The patient's levels are then maintained by an oral dosage form, although other forms of administration, dependent upon the patient's condition and as indicated above, can be used. The quantity to be administered will vary for the patient being treated.

Additionally, there is provided a kit for enhancing an immune response in a mammal comprising an inosine-containing compound, wherein said inosine-containing compound increases maturation of dendritic cells in order to enhance the immune response in a mammal thereof.

Finally, there is provided a composition for in vivo or ex vivo maturation of dendritic cells including an inosine-containing compound. The inosine-containing compound is any compound that includes an inosine molecule, one possible structure being defined as an oligonucleotide IpR having an oligonucleotide sequence (hereinafter, “R”) bonded to an inosine molecule (hereinafter, “I”) through a phosphate bond (hereinafter, “p”). More specifically, the oligonucleotide IpR has the following formula:
5Rₙ—p—l—p—Rₘ3′
wherein, I=an inosine molecule including, but not limited to, isoprinosine, inosine 5′-monophosphate, methyl inosine, 5′-monophosphate (MIMP), polymers

such as dimers and trimers, homologues thereof, and derivatives thereof;
p=a phosphate bond;
R=is an oligonucleotide sequence including at least
   two nucleotides including, but not limited to, C, T,
   A, and G;
n=is an integer from 0 to 100; and
m=is an integer from 0 to 100, wherein n plus m is greater than or equal to 1.

The oligonucleotide sequence (R) can be modified. For instance, in some embodiments, at least one nucleotide has a phosphate backbone modification. The phosphate backbone modification can be a phosphorothioate or phosphorodithloate modification. In some embodiments the phosphate backbone modification occurs on the 5′ side of the oligonucleotide or the 3′ side of the oligonucleotide. The oligonucleotide sequence (R) can be any size. Preferably the oligonucleotide has 2 to 150 molecules.

For use in the present invention, oligonucleotides can be synthesized de novo using any of a number of procedures well known in the art. For example, the β-cyanoethyl phosphoramidite method (S. L. Beaucage and M. H. Caruthers, (1981) Tet. Let. 22:1859) and the nucleoside H-phosphonate method (Garegg et al., (1986) Tet. Let. 27:4051-4054; Froehler et al., (1986) Nucl. Acid. Res. 14:5399-5407; Garegg et al., (1986) Tet. Let. 27:4055-4058, Gafffney et al., (1988) Tet. Let. 29;2619-2622) can be utilized. These chemistries can be performed by a variety of automated oligonucleotide synthesizers available in the market. Alternatively, oligonucleotides can be prepared from existing nucleic acid sequences (e.g. genomic or cDNA) using known techniques, such as those employing restriction enzymes, exonucleases, and/or endonucleases.

For use in vivo, oligonucleotides are preferably relatively resistant to degradation (e.g. via endo- and exo- nucleases). Oligonucleotide stabilization can be accomplished via phosphate backbone modifications. A preferred stabilized oligonucleotide has a phosphorothioate-modified backbone. The pharmacokinetics of phosphorothioate ODN show that they have a systemic half-life of forty-eight hours in rodents and suggest that they may be useful for in vivo applications (Agrawal, S. et al. (1991) Proc. Natl. Acad. Sci. USA 88:7595). Phosphorothioates may be synthesized using automated techniques employing either phosphoramidate or H phosphonate chemistries. Aryl- and alkyl- phosphonates can be made e.g. (as described in U.S. Pat. No. 4,469,863); and alkylphosphotriesters (in which the charged oxygen moiety is alkylated as described in U.S. Pat. No. 5,023,243 and European Patent No. 092,574) can be prepared by automated solid phase synthesis using commercially available reagents. Methods for making other DNA backbone modifications and substitutions have been described (Uhlmann, E. and Peyman, A. (1990) Chem. Rev. 90:544; Goodchild, J. (1990) Bioconjugate Chem. 1:165).

For administration in vivo, oligonucleotides can be associated with a molecule that results in higher affinity binding to target cell (e.g., B-cell and natural killer (NK) cell) surfaces and/or increased cellular uptake by target cells. Oligonucleotides can be ionically, or covalently associated with appropriate molecules using techniques, which are well known in the art. A variety of coupling or cross-linking agents can be used (e.g., protein A, carbodiimide, and N-succinimidyl-3-(2-pyridyidithio) propionate (SPDP)). Oligonucleotides can alternatively be encapsulated in liposomes or virosomes using well-known techniques.

The composition including the oligonucleotide IpR can be combined with a pharmaceutical composition or formulation as set forth and described above.

The above discussion provides a factual basis for the utility of the present invention. The methods used with and the utility of the present invention can be shown by the following non-limiting examples and accompanying figures.

### EXAMPLES

### Experimental Design, Material, and Methods

Routes of administration include, but are not limited to, subcutaneous, intraperitoneal and oral administration of MIMP. As proven below, MIMP is active on the human M-DC lineage when monocyte-derived precursors are cultured in vitro in the presence of GM-CSF+IL-4. The activity of MIMP on DC maturation can be assessed using a well-defined panel of cell surface markers that have been used to distinguish among the subtypes and follow their developmental progression. Generation of M-DC can be achieved from normal PB mononuclear cells using previously described methods that are routinely performed (See below). Because of the strong impact of endotoxin on DC growth, low endotoxin levels are vigorously maintained in all experimental procedures.

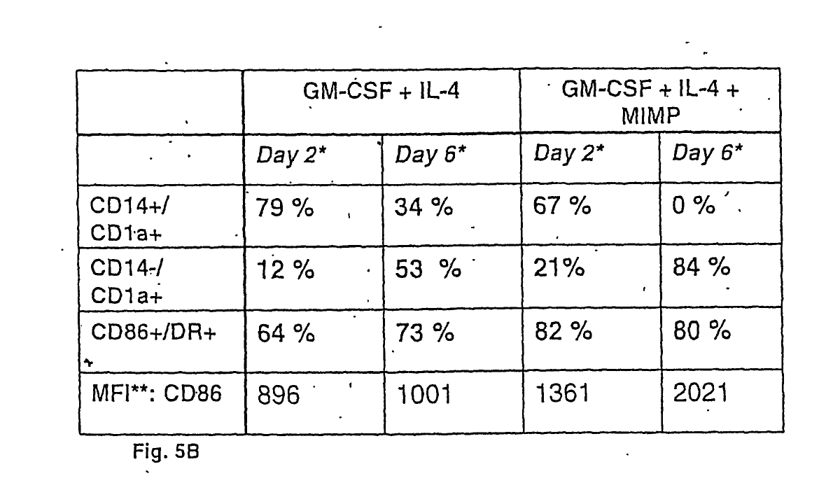

For obtaining monocyte-derived DCs, freshly isolated human PB mononuclear cells (PBMCs)(prepared by Ficoll density centrifugation) are adjusted to 5×10⁶ cells/ml in supplemented RPMI 1640 media containing 10% fetal calf serum, 2 mM L-glutamine, 10 mM HEPES, 50 IU/ml penicillin, and 50 μg/ml streptomycin (complete medium) and then adhered to plastic for 1 to 1.5 hours at 37° C. in a 5% CO₂ humidified incubator. After careful removal of any nonadherent cells, supplemented complete media containing human rGM-CSF (Genzyme) at 20-500 U/ml and human rIL-4 (Genzyme) at 500 U/ml were added. DC maturation inducing compounds, i.e. MIMP, TNFα,are added 24 hours after initial culture in GM-CSF+IL-4. All experimental procedures were performed under endotoxin poor conditions. Addition of TNFα served occasionally as a positive control for prototypical monocyte-derived DC maturation. MIMP is used at various doses (0.01-300 μg/ml or 0.01-300 mg/kg), added after 24 hours in GM-CSF and IL-4 alone. From the initial data (See, FIG. 6), MIMP is highly active at doses as low as 1 μg/ml.

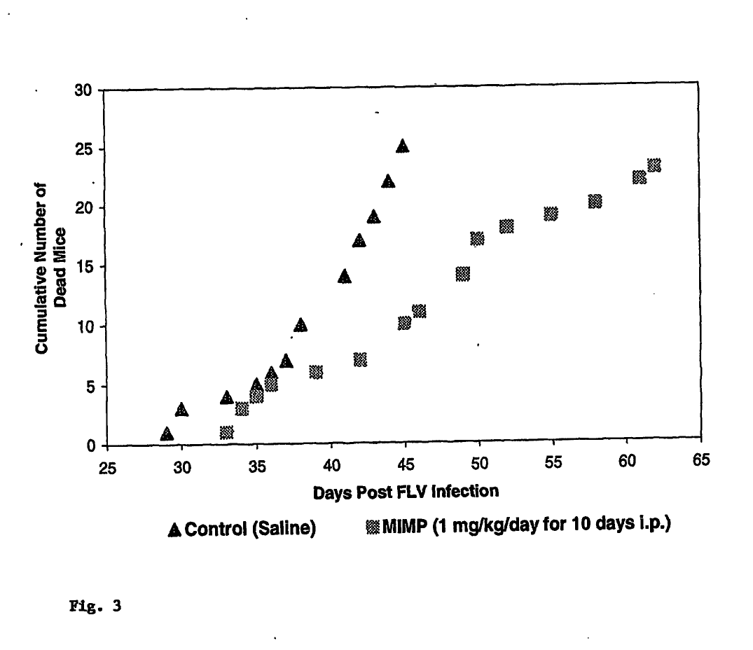

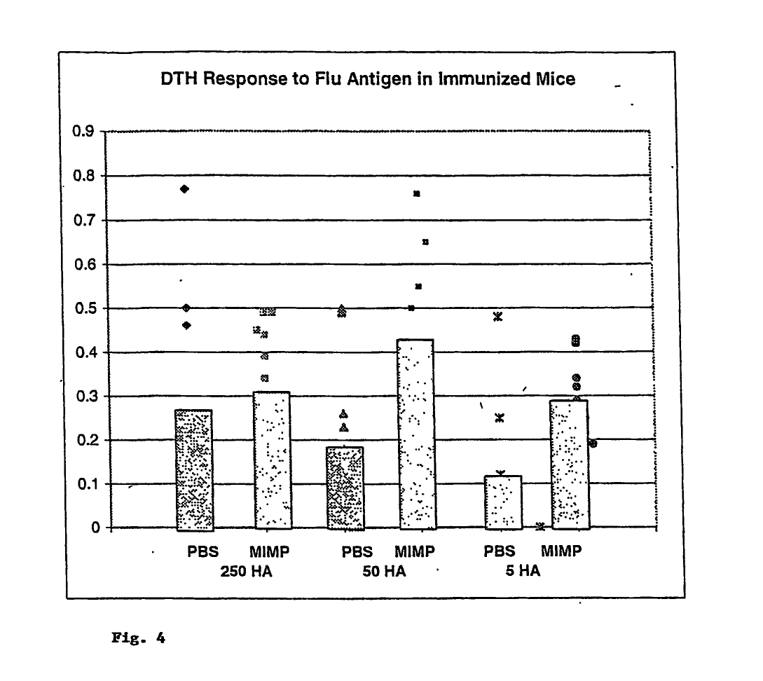

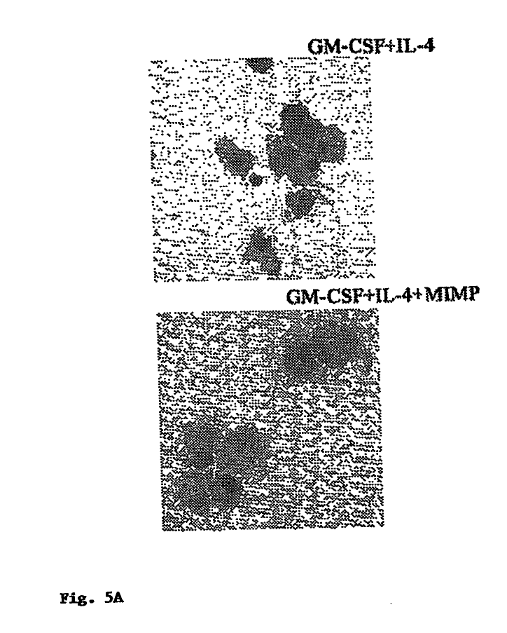

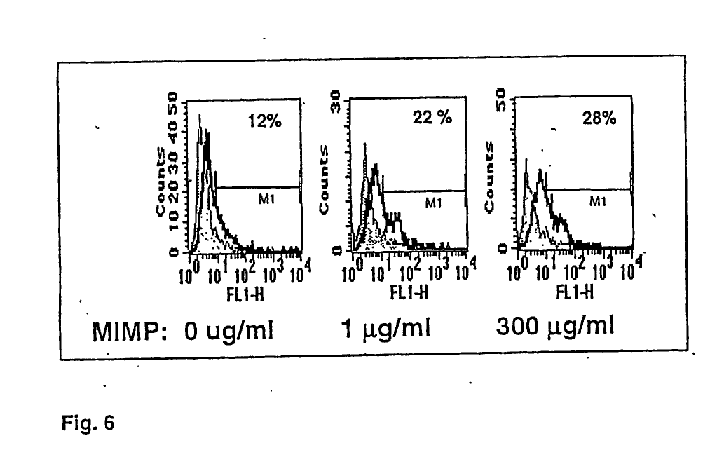

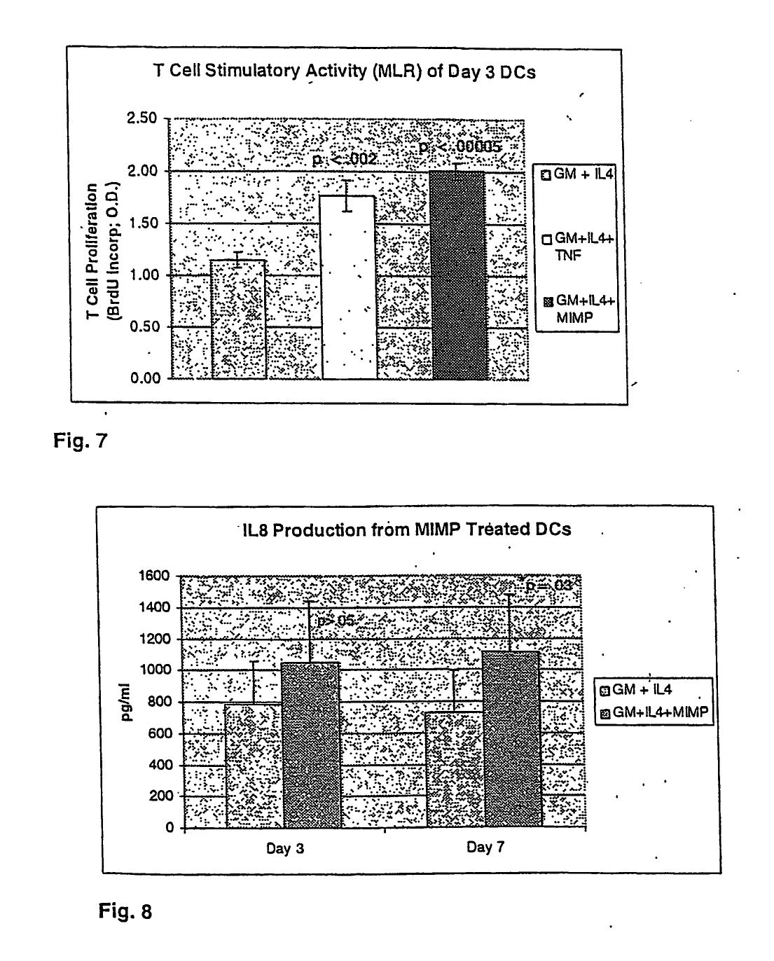

MIMP can accelerate DC maturation in vivo as evidenced by MIMP's general protective activity against viral and bacterial pathogens (FIGS. 2-3). MIMP also acts as an adjuvant to vaccines as shown by the ability to augment DTH responses to an influenza immunization (FIG. 4). In vitro, MIMP accelerated the conversion of M-DCs from an immature to a more mature morphology and cell surface marker expression (FIGS. 5 and 6). MIMP also enhances the functionality of DCs as demonstrated by the ability of MIMP-treated DCs to more effectively stimulate na [symbol] ve T cells and by the augmented production of the immune regulatory chemokine, IL8 (FIGS. 7 and 8).

### Example One

### In Vivo Protective Effects of MIMP against Bacterial and Viral challenge

In vitro and in vivo studies to date have shown that the immunostimulating activity of MIMP primarily targets T cell-dependent immune responses and preferentially enhances cell-mediated immune function. Such activity is consistent with MIMP stimulating and/or accelerating the maturation of dendritic cells. The overt consequences of enhanced cell-mediated immunity in an in vivo context are evidenced as protection against pathogenic challenges.

MIMP displayed protective effects in several in vivo models of infectious disease both pre- and post-exposure to pathogens following administration by one of several routes (i.e., intraperitoneal or oral). MIMP was tested in two lethal challenge mouse models with intracellular bacterial pathogens, Listeria monocytogenes and Salmonella typhimurium. Control animals were given doses of bacteria that caused rapid death with a mean survival time of 2.5 days and 100% mortality by day 4 or 5, respectively. As shown in FIG. 2, animals given MIMP intraperitoneally or in a combination of parenteral and oral administration starting five days prior to infection had an increased mean survival time (MST) and fully protected 40-50% of the animals challenged with Listeria. In the Salmonella model (data not shown), parenteral dosing of MIMP from 24 to only 4 hours prior to infection also resulted in prolongation of MST and protection of 10-20% of treated animals. A modest level of protection (10%) and MST extension were seen when MIMP was administered at four hours post-inoculation across the entire dose range from 0.1 mg/kg to 10 mg/kg.

MIMP demonstrated analogous protective activity against a viral challenge. As shown in FIG. 3, infection of mice with FLV (Friend Leukemia Virus) is rapidly lethal with a MST of thirty-nine days. In a stringent test of efficacy, initiating treatment at day three after infection, parenteral administration of MIMP (1 mg/kg/day) for ten days gave a 18% increase in MST to 46 days (See FIG. 3). It is noteworthy that a similar level of protection was observed when MIMP was administered via the drinking water.

The above evidence strongly supports a role for MIMP as a general immunostimulant and protective agent utilized with both pre- and post-pathogen (both viral and bacterial) exposure.

### Example Two

### MIMP Acts as an Adjuvant to Enhance Delayed Type Hypersensitivity (DTH) Responses to Vaccine antigens In Vivo

Adjuvants are defined as substances, which enhance the immune response to an admixed antigen over the response to antigen alone. Many classically defined adjuvants, e.g. mycobacteria in Freund's complete adjuvant, saponins, etc., have been identified as substances that activate and/or enhance the maturation of DCs. Similarly, MIMP acts as an adjuvant to vaccines as shown by the ability to augment in vivo immune responses to an influenza vaccination. FIG. 4 illustrates that MIMP combined with an immunizing antigen in this case inactivated (killed) influenza virus elicits increased T cell dependent responses in the form of delayed type hypersensitivity. Specifically, mice (Balb/c, 8 week old female) mice were immunized twice with either 250, 50, or 5 HA units per mouse (10 mice per group) of inactivated (formalin treated) mouse-adapted PR8 (H1N1) influenza virus, in either PBS or 1000 μg MIMP (approximately 30 mg/kg) (in a 0.2 ml volume total) on days 0 and 21 subcutaneously into the base of the tail. This was followed by a challenge inoculation of 200 HAU flu only, having no adjuvant, in the footpad 7 days after the booster injection. DTH swelling was measured 24 hours later. As shown in FIG. 4, MIMP enhance the DTH swelling response (vertical axis) at each dose of influenza virus. The most significant increases achieved by MIMP over antigen alone were found using doses of 50 and 5 HA flu antigen (p<0.05).

These data directly support the use of MIMP as an in vivo activator of DCs for augmenting immunological responses to various antigens, including those present in a vaccine. It also supports the use of MIMP in vivo for the treatment of disease, including infectious diseases.

### Example Three

### MIMP Causes Morphological Maturation of M-DC Precursonrs In Vitro

Several pilot studies looking at the effects of MIMP on human monocyte-derived DCs (M-DCs) have been achieved. As shown in the photographs (40× magnification of Wright stained cytospins) in FIG. 5A, MIMP treatment had a profound effect on the morphology of human M-DCs isolated from peripheral blood (PB). Adherent cells from a PB mononuclear cell preparation were cultured as described above in the presence of GM-CSF and IL-4, which promotes the development of “committed” but immature M-DCs (iM-DC). As shown in FIG. 5A, after 6 days in culture in the presence of GM-CSF+IL-4, these cells (left panel of FIG. 5A) have a relatively rounded appearance with few and short cellular processes. In contrast, after the same period in the presence of GM-CSF+IL-4+MIMP (5 days in the presence of MIMP; right photo panel of FIG. 5B) the cells have numerous and highly extended dendrite-like processes, which is characteristic of a mature DC phenotype. Intermediate changes in morphology due to MIMP treatment were also seen in as few as 2 days (24 hours of application of MIMP)(data not shown).

### Example Four

### MIMP Induces Expression of Cell-Surface Markers on M-DC Precursors Associated with Mature Phenotypes during In Vitro Culture

A further evaluation of in vitro MIMP-treated cells was performed by staining for CD14, CD1b/c, CD86 and HLA-DR and subsequent analysis by flow cytometry (See FIG. 5B). Human adherent mononuclear cells were prepared as previously described from peripheral blood and placed in media containing human rGM-CSF 20 U/ml) and human rIL-4 (500 U/ml). MIMP (300 μg/ml) was added 24 hours later. Two color immunofluorescence flow cytometry was performed as routinely described after 2 and 6 days in culture (24 hours and 5 days of MIMP treatment respectively).

FIG. 5B shows MIMP induced changes in cell surface markers associated with dendritic cell maturation on Day 2 and Day 6 of in vitro culture. Total culture time includes 24 hours in GM-CSF+IL-4 alone before the addition of MIMP, e.g. on Day 2 there was only 24 hours in the presence of MIMP. FIG. 5B shows that MIMP treatment increased the number of DR+/CD86+ cells and accelerated the loss of CD14+ (monocyte marker), i.e. decreased numbers of cells co-expressing CD14+ and CD1+ and increased numbers of cells expressing only CD1+ (single positive). There is also an increase in the mean fluorescence intensity of CD86 on the cells indicating that there is increased density of this co-stimulatory molecule on a given cell.

### Example Five

### MIMP Increases Expression of CD83, a Recognized Dendritic Cell Maturation Marker

The induction of the recognized dendritic cell maturation marker CD83 on human PB-derived M-DCs following MIMP treatment was also observed by flow cytometry. FIG. 6 illustrates that MIMP induces an approximately 2-fold increase in CD83 expression, wherein human peripheral blood adherent mononuclear cells were cultured as described for Example 4 and the indicated amount of MIMP was added to the cultures after one day of GM-CSF and IL-4 alone. The cells were stained with FITC-conjugated anti-human CD83, a maturation marker for dendritic cells, after a further forty-eight hour incubation. The cells were analyzed by flow cytometry as routinely described. The grey curve represents background staining with the isotype control and the M1 area indicates positive experimental values above background levels. The observation that MIMP increased CD83 expression at the lowest dose tested (1 μg/ml), suggests that even lower doses are active on dendritic cells.

Together these examples (FIGS. 5A, 5B and 6) provide evidence confirming that MIMP induces both morphological maturation and the expression of surface markers consistent with a mature DC phenotype.

### Example Six

### MIMP-Treated Dendritic Cells are Better Stimulators of Nave T Cells in Allogenic Mixed Lymphocyte Reactions (MLR)

As described earlier, the immature DC is designed to take up antigens but not to present them in an effective stimulatory fashion to na [symbol] ve T cells. Conversely, one of the defining functional characteristics of mature DCs is an ability to stimulate naive T cell responses. This capacity far surpasses that of other APCs, including monocytes and B cells and this is true for all DC lineages. The allogeneic mixed leukocyte reaction (MLR) assay remains the hallmark in vitro assay for assessing DC-mediated activation of na [symbol] ve T cells. Using such an assay, an examination of the functional consequences of MIMP treatment of PB-derived human M-DCs has occurred. In this experiment, MIMP-treated DC's (originating from human adherent peripheral blood mononuclear cell precursors) were assayed for their ability to stimulate allogeneic human T cells in a classical MLR. For the MLR, various doses of DCs are added to a fixed number of allogeneic T cells (nylon wool nonadherent T cells). DCs are harvested for use in the MLR after 3 or 7 days of standard in vitro culture (as described for Example 4) with GM-CSF (100-500 U/ml)+IL-4 (500 U/ml), with or without MIMP (300 μg/ml) (or TNFα(20 ng/ml)). After 6 days of co-incubation of the DCs with T cells in the MLR, T cell proliferation is measured by incorporation of bromodeoxyuridine (BrdU) via a colorimetric ELISA based assay. Responder cells (T cells) and stimulator cells (DCs) are incubated alone as background controls.

FIG. 7 shows the combined results of three independent experiments wherein the MIMP-incubated M-DCs are more effective at stimulating na [symbol] ve T cells than similar M-DCs treated with GM-CSF+IL4 alone or GM-CSF+IL4+ TNF α after 72 hours of total culture (48 hours in the presence of MIMP or TNFA). More effective stimulation is evidenced by the increased proliferation of the responding T cells, wherein the increase in effectiveness of added MIMP over either GM-CSF+IL-4 alone or GM-CSF+IL-4 + TNFα was highly significant (GM-CSF+IL-4 vs GM-CSF +IL-4 +MIMP: p<0.00005; GM-CSF+IL-4 +TNFa vs. GM-CSF+IL-4 +MIMP: p<0.002).

These results strongly support the claim that MIMP induces functional maturation of human PB-derived DCs, not just morphological and surface phenotypic changes. Such functional capabilities are well suited to treatment of conditions and diseases benefited by effective cell-mediated immune responses.

### Example Seven

### MIMP Enhances the Production of IL-8 from Dendritic Cells In Vitro

As dendritic cells are studied more closely, the functional capabilities of DCs have been revealed in increasing complexity. In addition to their T cell stimulatory functions, DCs also induce and polarize T cell responses by means of the cytokines and chemokines that they produce. They also use cytokines and chemokines to alert, activate and recruit other immune cells to sites of infection or disease.

FIG. 8 illustrates that MIMP treatment of human dendritic cells derived from adherent peripheral blood mononuclear cells, cultured in GM-CSF (20-500 U/ml)+IL-4 (500 U/ml)+MIMP (300 μg/ml) as described in Example 4, augments the production of chemoattractant IL8 above the amount made in the presence of GM-CSF and IL-4 alone. Supematants of in vitro cultures were harvested at day 3 and day 7 (2 days and 6 days respectively in the presence of MIMP) and assayed for the presence of IL-8 by standard ELISA methodology (R&D Systems). As shown in FIG. 8, which, represent the mean values +/× SEM from 7 independent experiments, there is an increased trend in IL-8 production in the MIMP treated DCs on day 3 and significant enhancement (p=0.03) by day 7 of in vitro culture.

These data demonstrate that MIMP in the presence of GM-CSF and IL4 augments the ability of DCs to secrete IL8, which is a known chemoattractant (chemokine) for a variety of cell types from both the innate and adaptive arms of the immune system.

The aforementioned examples provide factual evidence that the compositions and methods of the present invention are capable of increasing and enhancing the maturation of the dendritic cells in vivo and ex vivo. The inventive compositions are immunostimulants for use in maturing dendritic cells in vivo and ex vivo, either in combination with a specified antigen, for example as part of a vaccine or alone for stimulation with antigens, i.e. including but not limited to those present on the infectious agent or tumor cell. The examples provide specific data demonstrating that immune responses of a host mammal to combat pathogenic organisms are augmented by administering inosine-containing compounds. The data further demonstrates increased functional capabilities of dendritic cells by use of the present invention, including the enhanced stimulation of T cells in response to antigen and the enhanced production of immune-activating chemokine, IL-8.

Throughout this application, various publications, including United States patents, are referenced by author and year and patents by number. Full citations for the publications are listed below. The disclosures of these publications and patents in their entireties are hereby incorporated by reference into this application in order to more fully describe the state of the art to which this invention pertains.

The invention has been described in an illustrative manner, and it is to be understood that the terminology, which has been used, is intended to be in the nature of words of description rather than of limitation.

Obviously, many modifications and variations of the present invention are possible in light of the above teachings. It is, therefore, to be understood that within the scope of the described invention, the invention may be practiced otherwise than as specifically described.

### REFERENCES

1. Kelsall, B L et al., “Dendritic cells at the host-pathogen interface,” Na. Immunol, 3(8):699-702 (2002).
2. Steinman, R M et al., “Avoiding horror autotoxicus: the importance of dendritic cells in peripheral T cell tolerance,” Proc Natl Acad Sci USA, 99(1);351-358 (2002).
3. Sallusto, F. et al., “Chemokines and chemokine receptors in T-cell priming and Th1/Th2-mediated responses,” Immunol Today, 19(12):568-74 (1998).
4. Langenkamp, A. et al., “Kinetics of dendritic cell activation: impact on priming of TH1, TH2 and nonpolarized T cells,” Nat Immunol, 1(4):311-6 (2000).
5. Celia, M et al., “Plasmacytoid dendritic cells activated by influenza virus and CD401 drive a potent TH1 polarization,” Nat Immunol, 1(4):305-10 (2000).
6. Dzionek, A. et al., “Plasmacytoid dendritic cells: from specific markers to specific cellular functions(1), HumImmunol, 63(12):1133-48 (2002).
7. Zitvogel, L., “Dendritic and natural killer cells cooperate in the control/switch of innate immunity,” J Exp Med, 195(3):F9-14 (2002).
8. Santiago-Schwarz, F., “Positive and negative regulation of the myeloid dendritic cell lineage,” J Leukoc Biol, 66(2):209-216 (1999).
9. Strobl, H. et al., “Epidermal Langerhans cell development and differentiation,” Immunobiology, 198(5):588-605 (1998).
10. Gilliet, M. et al., “Human plasmacytoid-derived dendritic cells and the induction of T-regulatory cells,” Hum Immunol, 63(12):1149-1155 (2002).
11. Briere, F. et al., “Origin and filtration of human plasmacytoid dendritc cells,” Hum Immunol, 63(12):1081-1093 (2002).
12. Dzionek, A. et al., “BDCA-2, BDCA-3, and BDCA-4: three markers for distinct subsets of dendritic cells in human peripheral blood,” J Immunol, 165(11):6037-6046 (2000).
13. Kadowaki, N. et al., “Natural type 1 interferon-producing cells as a ling between innate and adaptive immunity,” Hum Immunol, 63)12):1126-32 (2002).
14. Teixeira, M M et al., “Introduction: innate recognition of bacteria and protozoan parasites,” Microbes Infect, 4(9):883-886 (2002).
15. Akira, S. et al., “Toll-like receptors: critical proteins linking innate and acquired immunity,” Nat Immunol, 2(8):675-80 (2001).
16. Kadowaki, N. et al., “Subsets of human dendritic cell precursors express different toll-like receptors and respond to different microbial antigens,” J Exp Med., 194(6): 863-9 (2001).
17. Hemmi, H. et al., “A Toll-like receptor recognizes bacterial DNA,” Nature, 408(6813):740-5 (2000).
18. Hemmi, H. et al., “Small anti-viral compounds activate immune cells via the TLR7 MyD88-dependent signaling pathway,” Nat Immunol., 3(2):196-200 (2002).
19. Srivastava, P., “Roles of heat-shock proteins in innate and adaptive immunity,” Nat Rev Immunol., 2(3):185-194 (2002).
20. Bendelac, A. et al., “Adjuvants of immunity: harnessing innate immunity to promote adaptive immunity,” J Exp Med., 195(5):F19-23 (2002).
21. Gibson, et al., “Plasmacytoid dendritic cells produce cytokines and mature in response to the TLR7 agonists, imiquimod and resiquimod,” Cell Immunol., 218(102):74-86 (2002).
22. Rothenfusser, S. et al., “Plasmacytoid dendritic cells: the key to CpG(1),” Hum Immunol., 63(12):1111-1119 (2002).
23. Dzionek, A. et al., “BDCA-2, a novel plasmacytoid dendritic cell-specific type II C-type lectin, mediates antigen capture and is a potent inhibitor of interferon (α/β induction,” J Exp Med., 194(12):1823-34 (2001).
24. Hirao, M. et al., “CC chemokine receptor-7 on dendritic cells is induced after interaction with apoptotic tumor cells: critical role in migration from the tumor site to draining lymph nodes,” Cancer Res., 60(8):2209-17 (2000).
25. Medzhitov, R. and C. A. Biron, “Innate Immunity,” Curr Opin Immunol., 15(1): p. 2-4 (2003).
26. Palucka, K. and J. Banchereau, “Dendritic cells: a link between innate and adaptive immunity,” J. Clin. Immunol., 19(1): p. 12-25 (1999).
27. Shortman, K. and Y. J. Liu, “Mouse and human dendritic cell subtypes,” Nat Rev Immunol., 2(3): p. 151-61 (2002).

What is claimed is:

1. A method of stimulating maturation of dendritic cells in vivo or ex vivo by applying an effective amount of inosine-containing compounds to the dendritic cells.

2. The method according to claim 1, wherein said applying step is defined as applying an inosine-containing compound selected from the group consisting of isoprinosine, inosine 5′-monophosphate, methyl inosine 5′-monophosphate (MIMP), polymers thereof such as dimers and trimers, oligonucleotides including one or more inosine 3′, 5′ linkages, homologues thereof, and derivatives thereof.

3. The method according to claim 2, wherein said applying step is further defined as applying the inosine-containing compound for not less than twenty-four hours.

4. A method of treating diseases in a subject by applying an effective amount of an inosine-containing compound to the dendritic cells to stimulate maturation thereof; and administering matured dendritic cells into the subject.

5. The method according to claim 4, further including the step of fostering the secretion of cytokines and chemokines, which foster the development of Th1 responses in T cells.

6. A composition for maturing dendritic cells ex vivo for treatment of in vivo diseases.

7. The composition according to claim 6, wherein said composition is an inosine-containing compound.

8. The composition according to claim 7, wherein said inosine-containing compound is selected from the group consisting of isoprinosine, inosine 5′-monophosphate, methyl inosine 5′-monophosphate (MIMP), inosine-containing oligonucleotides, and polymers thereof such as dimers and trimers, oligonucleotides including one or more inosine 3′, 5′ linkages, homologues thereof, and derivatives thereof.

9. The composition according to claim 7, wherein said inosine-containing compound is in a dose range of approximately 0.01 to 300 mg/kg.

10. The composition according to claim 7, wherein said diseases are selected from the group consisting of cancer, immune deficiencies, and infectious diseases.

11. A pharmaceutical composition for enhancing in vivo dendritic cell function comprising an effective amount of an inosine-containing compound, wherein the enhanced dendritic cell function is utilized for treatment of subjects with diseases.

12. The pharmaceutical composition according to claim 11, wherein said inosine-containing compound is selected from the group consisting of isoprinosine, inosine 5′-monophosphate, methyl inosine 5′-monophosphate (MIMP), inosine-containing oligonucleotides, and polymers thereof such as dimers and trimers, oligonucleotides including one or more inosine 3′, 5′ linkages, homologues thereof, and derivatives thereof.

13. The pharmaceutical composition according to claim 12, wherein said inosine-containing compound is in a dose range of approximately 0.01 to 300 mg/kg.

14. An immunostimulant for use in a vaccine comprising an effective amount of an inosine-containing compound for use in maturing dendritic cells in vivo or ex vivo, wherein antigens are of low immunogenicity and multiple doses are required.

15. The immunostimulant according to claim 14, wherein said inosine-containing compound is selected from the group consisting of isoprinosine, inosine 5′-monophosphate, inosine-containing oligonucleotides, and methyl inosine 5′-monophosphate (MIMP), polymers thereof such as dimers and trimers, oligonucleotides including one or more inosine 3′, 5′ linkages, homologues thereof, and derivatives thereof.

16. The immunostimulant according to claim 15, wherein said inosine-containing compound is in a dose range of approximately 0.01 to 300 mg/kg.

17. An adjuvant to be used with vaccines comprising an effective amount of an inosine-containing compound for use in maturing dendritic cells.

18. The adjuvant according to claim 17, wherein said inosine-containing compound is selected from the group consisting of isoprinosine, inosine 5′-monophosphate, methyl inosine 5′-monophosphate (MIMP), polymers thereof such as dimers and trimers, oligonucleotides including one or more inosine 3′, 5′ linkages, homologues thereof, and derivatives thereof.

19. The adjuvant according to claim 17, wherein said inosine-containing compound is in a dose range of approximately 0.01 to 300 mg/kg.

20. A method of stimulating maturation of dendritic cells in vitro by applying an effective amount of an inosine-containing compound to the dendritic cells and increasing dendritic processes, expressing appropriate cell surface markers on cell surface of dendritic cells, and increasing functionality of the dendritic cells thereof.

21. The method according to claim 20, wherein said applying step is further defined as applying an inosine-containing compound selected from the group consisting of isoprinosine, inosine 5′-monophosphate, methyl inosine 5′-monophosphate (MIMP), inosine-containing oligonucleotides, and polymers thereof such as dimers and trimers, oligonucleotides including one or more inosine 3′, 5′ linkages, homologues thereof, and derivatives thereof.

22. A kit for enhancing an immune response in a mammal comprising an effective amount of an inosine-containing compound in a dose range of approximately 0.01 to 300 μg/ml for increasing maturation of dendritic cells in order to enhance the immune response in a mammal thereof.

23. The kit according to claim 22, wherein said inosine-containing compound is selected from the group consisting of isoprinosine, inosine 5′-monophosphate, methyl inosine 5′-monophosphate (MIMP), inosine-containing oligonucleotides, and polymers thereof such as dimers and trimers, oligonucleotides including one or more inosine 3′,5′ linkages, homologues thereof, and derivatives thereof.

24. A method of enharicing the immune response of a host mammal by isolating immature dendritic cells from a donor mammal; maturing the immature dendritic cells in the presence of an effective amount of an inosine-containing compound, either in the presence or absence of antigen; and administering the mature dendritic cells to a host mammal in an amount effective to enhance the immune response of the host mammal.

25. The method according to claim 24, further including the step of loading the immature dendritic cells with antigens.

26. A method increasing the T cell stimulatory activity of dendritic cells by maturing dendritic cells in the presence of an inosine-containing compound.

27. The method according to claim 26, wherein said maturing step is defined by increasing expression of cell surface markers.

28. The method according to claim 27, wherein said increasing step is further defined as expressing cell surface markers selected from the group consisting of CD1a-c, CD11c, CD14, CD40, CD80, CD83, CD86, CD123, HLA-DR, BDCA-2, BDCA-4, Toll-like receptors (TLR), heat shock protein receptors (CD91), scavenger receptors, mannose receptors, complement receptors, and lectin receptors.

29. A composition for enhancing in vivo or ex vivo maturation of dendritic cells comprising an inosine-containing compound.

30. The composition according to claim 29, wherein said inosine-containing compound is defined as an oligonucleotide IpR including an oligonucleotide sequence (R) bonded to an inosine molecule (I) through a phosphate bond (p).

31. The composition according to claim 30, wherein said oligonucleotide IpR has the formula:
5′Rₙ—p—l—p—Rₘ3′
wherein, I=an inosine molecule selected from the group consisting of isoprinosine, inosine 5′-monophosphate, methyl inosine
5′-monophosphate (MIMP), homologues thereof, and derivatives thereof;
p=a phosphate bond;
R=is an oligonucleotide sequence including at least one nucleotides selected from the group consisting of C, T, A, and G;
n=is an integer from 0 to 100; and
m=is an integer from 0 to 100, wherein n plus m is greater than or equal to 1.

32. The composition according to claim 30, wherein said oligonucleotide has about 2-150 nucleotides.

33. A method of stimulating maturation of dendritic cells of an individual in vivo by administering an effective amount of inosine-containing compounds to the individual.

34. A method of inducing immune regulatory cytokines and chemokines by exposing dendritic cells to an inosine-containing compound.

35. A method as set forth in claim 34, wherein the dendritic cells are of human origin.

36. A composition for inducing immune regulatory cytokines and chemokines comprising an inosine-containing compound.

37. A method of enhancing stimulatory activity of dendritic cells to T cells by administering an effective amount of inosine-containing compounds thereby leading to a more robust cellular immune response against antigens.

38. A method of enhancing a response to vaccine antigens by administering a combination of antigen with an inosine-containing compound in the form of a vaccine, wherein the inosine-containing compound is considered as an adjuvant.

39. An adjuvant comprising an inosine-containing compound and antigen.

40. A method for enhancing immunological responses to vaccines by administering a dendritic cell stimulating adjuvant.

41. A method of increasing presentation of antigens to T cells by maturing dendritic cells in the presence of an inosine-containing compound for stimulating the presentation of antigen to the T cells.

42. A composition comprising dendritic cell stimulating means for stimulating the presentation of antigen to T cells.

43. A composition as set forth in claim 42, wherein said dendritic cell stimulating means includes an inosine-containing compound.
